(19) United States
(12) Patent Application Publication
(10) Pub. No.: US 2003/0044849 A1
(43) Pub. Date: Mar. 6, 2003

(54) Methods for screening monoclonal antibodies on heterogeneous antigen substrates

(76) Inventor: Steven Kessler, Belmont, CA (US)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/225,371

(22) Filed: Aug. 21, 2002

Related U.S. Application Data
(60) Provisional application No. 60/314,070, filed on Aug. 22, 2001.
(60) Provisional application No. 60/314,071, filed on Aug. 22, 2001.

(51) Int. Cl. G01N 33/53; C12P 21/04; C12N 5/06

(52) U.S. Cl. 435/7.1; 435/70.21; 435/326

(57) ABSTRACT

Methods, compounds, and polyclonal antibody libraries (PALs) that relate to screening and selection of antibodies and the cells that produce them are disclosed. In particular, an antigen or a subset of antigens which has some desired feature can be distinguished from among a multitude of other undesired antigens present in a heterogeneous antigen mixture which lack such feature by use of PALs raised against the undesired antigens. When such PALs are brought into contact with a heterogeneous antigen substrate, the antibodies in the PAL can bind to those antigens, masking them from subsequent binding by a test antibody having a similar specificity. By decreasing binding of test antibodies to those undesired antigens, if binding of test antibodies to the substrate occurs, such binding will more likely be by way of binding of the antibody to a desired antigen in the mixed antigen substrate. Thus, this invention can improve the efficiency of screening antibodies directed towards desired antigens. By appropriate selection of a PAL, effects such as false positives can be decreased, even for unknown undesirable antigens.

### RELATED APPLICATION

This application claims priority under 35 U.S.C. §119 to U.S. Provisional Patent Applications Serial No. 60/314,070 filed Aug. 22, 2001 and Serial No. 60/314,071 filed Aug. 22, 2001 and is related to United States Utility Patent Application titled “Methods for Screening Antibody-Producing Cells on Heterogeneous Antigen Substrates” Steven Kessler, inventor, Attorney Docket No. KSLR 1001 US1 SRM/DBB, filed concurrently. Each of the above-identified patent applications is incorporated herein fully by reference.

### BACKGROUND OF THE INVENTION

1. Field of the Invention

This invention relates to the screening and production of monoclonal antibodies and phage antibodies for binding to a molecular target. Specifically this invention relates to using a library of polyclonal antibodies directed against a number of undesirable antigens to mask those antigens so that monoclonal antibodies directed toward desirable antigens may be more efficiently screened.

2. Description of Related Art

The value of monoclonal antibodies as therapeutic immune effector and drug delivery agents, as tools for in vitro disease diagnosis and in vivo disease imaging, and as tools for discovery of new drugs continues to rise. Early clinical studies have shown monoclonal antibodies to produce durable responses in several solid cancers, and partial alleviation of symptoms in certain autoimmune and inflammatory diseases. Used as diagnostic and imaging tools, they may prove invaluable for staging malignancies, for monitoring the progression of disease and responses to therapy, and for the development of more patient-specific therapies. Used in conjunction with genomics-based and proteomics-based or protein biochip array approaches, monoclonal antibodies may play key roles in discovery of molecular pathways and drug targets. In view of these applications there is much interest in the continuing discovery of monoclonal antibodies to additional antigenic biomarkers, and especially on the surfaces of cells associated with disease processes.

Monoclonal antibodies are antibodies obtained from a population of substantially homogeneous cells cloned from a common parent cell. The individual antibodies derived from a single clone are identical except for possible naturally occurring mutations that may be present in minor amounts. Monoclonal antibodies are highly specific, being directed against a single antigenic determinant (“epitope”). In contrast to conventional (polyclonal or oligoclonal) antibody preparations, which typically include different antibodies directed against different antigens or determinants, each monoclonal antibody is directed against a single determinant on the antigen. Thus, monoclonal antibodies typically consist of a plurality of antibodies directed against the same epitope. The modifier “monoclonal” as used herein indicates the character of the antibody as being obtained from a substantially homogeneous population of cells, and is not to be construed as requiring production of the antibody by any particular method. For example, the monoclonal antibodies to be used in accordance with the present invention may be made by hybridoma methods first described by Kohler et al., Nature 256:495 (1975), incorporated herein fully by reference, or its subsequent modifications, or may be made by recombinant DNA methods. Monoclonal antibodies may also be isolated from phage antibody libraries generated using methods such as those described by McCafferty and Johnson (in: Phage Display of Peptides and Proteins; Kay, Winter and McCafferty, eds., Academic Press 1996, pp. 79-111), incorporated herein fully by reference.

In hybridoma methods, a mouse or other appropriate animal, such as a hamster, rat, rabbit, cow, sheep, monkey or human, and the like is immunized with antigen to elicit lymphocytes that produce or are capable of producing antibodies that will specifically bind to the antigen used for immunization. Alternatively, lymphocytes may be immunized in vitro. Lymphocytes are then fused with parental myeloma cells using a suitable fusing agent, such as polyethylene glycol, to form a hybridoma that has immortal growth potential. Preferred myeloma cells include those that fuse efficiently, support stable high-level production of antibody by the selected antibody-producing cells, and/or are sensitive to a drug selection medium. Among these, preferred mycloma cell lines include murine myeloma lines such as SP2/0. Human myeloma (Karpas A, et al., Proc Natl Acad Sci U S A (2001) 98:1799) and mouse-human heteromyeloma (Arinbjamarson S and Valdimarsson H (2002) J Immunol Methods (2002) 259:139) cell lines also have been described for the production of human monoclonal antibodies. A rabbit myeloma cell line has been described for producing rabbit monoclonal antibodies (Spieker-Polet H, et al., Proc Natl Acad Sci USA (1995) 92: 9348). It can be appreciated that any type of myeloma cell lines that can fuse efficiently with lymphocytes can be advantageously used with the methods and compositions of this invention.

The hybridoma cells thus obtained are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Importantly, seeding of the hybridoma cells is generally performed at a suitably low cell density, most commonly in 96-well microtiter plates or trays, so that after drug selection each well preferably contains no more than one surviving hybridoma clone. The culture medium or supernatant from each well in which hybridoma cells are growing is then assayed or screened individually for production of monoclonal antibodies directed against the antigen. This typically requires the collection and screening of culture medium from hundreds to several thousands of hybridoma clones from a single mouse hybridoma fusion experiment.

Also importantly, the binding specificity of monoclonal antibodies produced by the hybridoma cells is determined by any of a variety of in vitro binding assays, depending on the nature of the antigen. For example, when a defined or purified antigen is available a radioimmunoassay (RIA) or enzyme-linked immunoabsorbent assay (ELISA) may be used.

By contrast, when an antigen is present in a heterogeneous antigen mixture or is undefined, as for example might be encountered with intact cells or cell extracts or fractions, the binding specificity is commonly determined by immunocytochemical or immunohistochemical staining using flow cytometry or microscope imaging analysis. In the latter situation, there may be a multi-stage screening process, first, a semi-specific test for antibody production (i.e., positivity), followed by broader analysis of specificity.

Further determination of the specificity of each monoclonal antibody to a cell-associated antigen typically involves a comparative analysis of staining of a few to several dozen or more different cell types or tissue specimens in order to discern a pattern or range of antigen distribution. Thus, the total number of individual data determinations is some multiple of the original number of positive hybridomas. The complexity of this process can pose a burden to the economics of labor, time and costs of screening efforts. The use of automation, robotics and optical imaging have made the latter process somewhat less tedious, but the logistics of generating and interpreting large numbers of data determinations, whether positive or negative, represent significant rate-limiting steps in high-throughput screening efforts.

After hybridoma cells are identified that produce antibodies of the desired specificity, affinity, and/or activity, the clones may be subcloned by limiting dilution procedures and grown by standard methods in suitable culture medium. In addition, the hybridoma cells may be grown in vivo as ascites tumors in an animal. The monoclonal antibodies secreted by the subclones are then suitably separated from the culture medium, ascites fluid, or serum by conventional immunoglobulin purification procedures such as, for example, Protein A- or Protein G-Sepharose chromatography, hydroxylapatite chromatography, ion exchange chromatography, gel electrophoresis, dialysis, or affinity chromatography. Protein A or G chromatography is particularly useful because it retains the ability to bind proteins (i.e., immunoglobulins) from most mammalian species which have a C_{H}2/C_{H}3 region.

### SUMMARY OF THE INVENTION

This invention includes compositions, methods and kits suitable for detecting desired antigens that are present within a mixture of both desirable antigens and undesirable antigens. Libraries comprising antibodies directed towards undesirable antigens are used to mask a mixed antigen substrate so that desirable antigens remain exposed for subsequent binding to test antibodies. Binding of test antibodies to a masked antigen substrate increases the likelihood that the test antibody recognizes a desirable antigen on the substrate. A library or libraries of polyclonal antibodies (“polyclonal antibody library(ies)” or “PAL(s)”) is prepared against heterogeneous mixtures of antigens. The antigens can be derived from intact cells, cell extracts, cellular organelles, cellular molecules, cellular fractions, cellular digests, or other cellular components. Cells can be either naturally occurring, or can be transgenic or recombinant. For example, cells can be made from subtracted DNA libraries that have a subset of normal cellular constituents. A heterogeneous mixture of antigens derived from a cell type not having a desired antigen can be used as an immunogen. Lymphocytes isolated from animals having such an immunization can be fused with myeloma cells, or can be immortalized using other methods to provide a mixed pool of renewable antibody producing cells. This mixed population of cells can be expanded and grown to produce a large, mixed population of antibody producing cells, each of which produces antibodies directed against a certain set of antigens, including previously unknown and/or undetected antigens. Antibodies produced by such populations of cells can be collected into a PAL. The libraries so produced can be designed not to contain an antibody directed against an antigen of interest. It can be appreciated that a PAL may comprise IgG, IgE, IgM or any other type of antibody or antibody fragment. Thus, the discussion of IgG producing cells is not intended to be limiting to the scope of this invention.

A PAL can then used as a mask to block those antigenic sites on a target that are recognized by antibodies within the pool. For example, a target may be a specific tumor cell. If a PAL is prepared against antigens from normal or non-tumorous cells, then the components of the PAL can bind to and “mask” those antigens if they are present in the tumor cell. Thus, non-normal or unique tumor antigens will be unoccupied in the heterogeneous antigen substrate, and a test monoclonal antibody, if it binds to the substrate, is more likely to recognize a non-normal antigen, thereby decreasing the probability of “false positives” in a screening assay for monoclonal antibodies directed toward such unique antigens. Such masked antigen substrates then can provide a means for evaluating test monoclonal antibodies for their ability to recognize antigens not present in normal cells. Any test monoclonal antibodies that are directed toward antigens present on normal cells will therefore not be bound, because antibodies from the PAL will have already substantially bound to those antigens and thereby block subsequent binding of a monoclonal antibody. Therefore, any binding of test monoclonal antibodies to a PAL pre-treated antigen substrate will reflect the binding to a non-normal antigen.

Polyclonal antibody libraries of this invention and methods by which they are produced can provide significant advantages over conventional antibody preparations obtained from antiserum. One advantage is that PALs of this invention are substantially comprised of antibody molecules directed toward the various antigens in the immunogen because the antibodies are derived from selected lymphoid organs (e.g., spleen and/or peripheral lymph nodes). Those organs and tissues contain antigen-specific B-lymphocytes that are in a proliferating state after recent immunization. When these proliferating cells are immortalized, they typically continue to produce antibodies to the same antigens that they responded to in the immunogen. Thus, the immortalization process can be relatively selective for specific antibody production that were produced in response to the initial immunization. By contrast, specific antibodies to a given immunogen in conventional antiserum generally represent a minority of the total population of antibodies present in the antiserum, rarely reaching more than about 5% to about 10% of the total numbers of immunoglobulin molecules, and mostly are present in even smaller amounts. One technique that has been used to enrich specific antibodies from antiserum is affinity purification. However, affinity purification typically requires a known antigen affixed to an affinity matrix. Affinity purification of specific antibodies in antiserum is difficult to carry out when it is desirable to have a large number of different antibodies directed toward a variety of different, possibly undefined antigens on a scale needed for desired purposes, such as antigen masking.

Another desirable feature of the methods and PALs of this invention is that, unlike conventional mixtures of monoclonal antibodies, the antibody libraries of this invention need not be directed to pre-determined antigens. It is not necessary to predefine the individual antibody specificities of each component of the PAL.

Polyclonal antibody libraries can be relatively devoid of extraneous and/or undesired antibodies that are typically found in polyclonal antisera. Undesired antibodies include high levels of so-called “natural antibodies” with wide-ranging or widely cross-reactive specificities to antigens in the environment, cells from other species, or to intestinal flora of the animal used for producing antisera. In some cases, these cross-reactive specificities include antigens of mammalian cells, which could be undesirable. For example, a significant proportion of natural antibodies or undesired antibodies may be produced by lymphoid tissues associated with the gut or bone marrow, which are not typically harvested for the purpose of making hybridomas. Removal of these undesired antibodies typically requires absorption of antiserum with large amounts of animal tissue, which may be impractical with large fluid volumes and may introduce contaminants into the antiserum. In contrast, PAL of this invention contain antibodies more likely to be directed toward antigens present in the immunogen.

It can be appreciated that aPAL of this invention can be easily renewed or replenished in the form of immortalized, permanent lines including hybridomas. This replenishment can be accomplished without additional immunizations. In contrast, polyclonal antiserum derives from a single immunized animal or group of animals each producing an individual spectrum of antibodies that may not be reproducible from one animal to the next, or even in the same animal over time. Collection of polyclonal antiserum is thus limited in volume and limited by the useful lifespan of the animal. Moreover, even if a moderate amount of antiserum is collected, this may lose potency with time in storage. Replenishment of a PAL in the form of immortalized lines can thus have significant savings over polyclonal antiserum with respect to reproducibility, time, and quantities of immunogen needed (which could be in limiting supply in some circumstances).

Polyclonal antibody libraries of this invention can be well suited to screening studies of monoclonal antibodies to be used, for example, for antigen discovery, diagnostic and/or therapeutic purposes, among others.

Kits comprising a PAL(s), PAL producing cells, heterogeneous antigen substrates derived from desired targets, and methods of using those compositions and kits can allow desired monoclonal antibodies directed at novel antigens to be discovered with substantially greater efficiency.

Thus, in one aspect, the invention includes methods for preparing heterogeneous antigen mixtures to solid substrates for the purpose of reacting those antigen mixtures with a PAL.

In another aspect, the invention includes methods for tailoring the specificities of a PAL so that the antibodies can bind to a majority of different antigens on a substrate except for those antigens having a desired property (e.g., are tumor-cell specific).

Another aspect encompasses methods for using PALs for binding or blocking various antigens on a substrate.

In yet another aspect, this invention includes methods for preparing PALs so that they lack an antigenic or structural feature that may be present on a test monoclonal antibody. Such structural or antigenic differences can permit test monoclonal antibodies to be distinguished from the PAL antibodies bound to the substrate.

Yet further aspects include methods for maintaining, replenishing, and/or expanding PALs.

Still other aspects include compositions of matter comprising PALs and/or mixed pools of monoclonal cells that produce a PAL, including uncloned or cloned, and immortalized cell lines, including hybridomas, transfectomas or bacteriophage that are sources of PALs.

Additional aspects include PALs having specificities tailored for antigens that are structurally distinguishable from antigens against which test antibodies are prepared.

In still further aspects, this invention includes methods to facilitate the screening and production of monoclonal antibodies directed toward specific, desired antigens that may be present in homogeneous or heterogeneous form or in mixtures of antigens from specific target cells, cell populations, cellular organelles, digests, tissues, organs or organisms.

The compositions and methods of this invention can be used to reduce the complexity and time necessary for generating, screening and selecting antibody-producing cells compared to conventional methods. One application of the compositions and methods of this invention is in high throughput screening of monoclonal antibodies for antigen discovery and/or therapy using monoclonal antibodies as therapeutic agents or to target therapeutic agents to specifically desired cell types.

### BRIEF DESCRIPTION OF THE DRAWINGS

FIG. 1 is a scheme of the different consequences of reacting a PAL having specificities for various antigens on a heterogeneous antigen substrate on the subsequent binding of different monoclonal antibodies to different antigens in the antigen substrate.

FIG. 2 is a scheme of the composite specificities of PAL and monoclonal antibodies raised to heterogeneous antigen mixtures, illustrating specificities that are overlapping and specificities that are unique to each population of antibodies.

FIG. 3a depicts the non-covalent binding of PAL to antigens present in a heterogeneous antigen substrate having a target antigen not recognized by antibodies in the PAL.

FIG. 3b depicts the PAL and heterogeneous antigen substrate as in FIG. 3a after addition of a monoclonal antibody directed toward the target antigen.

FIG. 4a depicts another embodiment of this invention in which a PAL directed toward antigens present in a heterogeneous antigen substrate is covalently attached to those antigens against which antibodies in the PAL are directed and a target antigen not recognized by antibodies in the PAL.

FIG. 4b depicts the PAL and heterogeneous antigen substrate as depicted in FIG. 4a after addition of a monoclonal antibody directed toward the target antigen.

### DETAILED DESCRIPTION

The methods provide for the production of renewable libraries comprised of soluble polyclonal or oligoclonal antibody mixtures with multiple specificities directed toward a heterogeneous subset of antigens present in or on intact cells, cell extracts, cell fractions, cellular organelles, and/or digests. For convenience of reference, such an antibody library is herein termed a “PAL.” This term is meant to be used in the broadest sense consistent with its composition and use, and should not be construed as limiting its use for any specific application. Other terms may be defined that are equally descriptive of the same compositions. The term “antibody” is used here in the broadest sense and specifically covers polyclonal antibodies, monoclonal antibodies (including full-length monoclonal antibodies), multispecific antibodies (e.g., bispecific antibodies), and single domain antibodies, phage antibodies and antibody fragments. The terms “specificity” and “specificities” mean the degree to which a given antibody or mixture of antibodies react with certain antigens. Thus, mixtures of antibodies with overlapping specificities may react with a number of different antigens in common, yet one mixture still may react with antigens not recognized by other mixtures of antibodies.

As used herein, the term “antigen” includes meanings known in the art, and means a molecule or portion of a molecule that can react with a recognition site on an antibody. The term “antigen” also includes a molecule or a portion of a molecule that can, either by itself or in conjunction with an adjuvant or carrier, elicit an immune response (also called an “immunogen”).

Included in the term “intact cells” as used herein include eucaryotic or procaryotic cells that may naturally express, or may be genetically modified to express, or may be coupled with, any antigen or antigens of interest. The term “cell extracts or fractions” broadly includes cell-derived organelles, proteins, glycoproteins, glycolipids, nucleic acids and other cellular components or antigens. Any of these may be extracted using aqueous, detergent or organic solvents, freeze-thawing, sonication, cavitation, secretion, shedding, enzymatic digestion or other methods, and may be fractionated by various methods such as by size, solubility, density, charge, affinity or chemical labeling, antibody or lectin binding, chemical or enzymatic fragmentation, differential display or expression, molecular cloning efficiency or mutagenesis, etc. Any of these above materials may be used as an immunogen.

The terms “heterogeneous antigen” and “heterogeneous antigen mixture” includes a mixture of two or more antigens used for immunization or detection (e.g., screening). The term “heterogeneous antigen substrate” includes a mixture of two or more antigens derived from a source and are applied to a detection surface, such as, by way of example, a slide, wafer, dish, and the like. The term “antigen” as used herein includes molecules or portions of molecules (epitopes) that can elicit production of antibodies or that can bind to antibodies. The term includes materials that react strongly and with high specificity, and also includes materials that react weakly and/or with low affinity to an antibody.

The intact cells, cell extracts or fractions, tissue sections or the like can be attached to a surface or matrix by suitable means to produce an antigen substrate. The surface or matrix may be any material known in the art to adhere to antigens sufficiently to remain adhered even after being subjected to conditions of washing, with solutions necessary for analysis by antigen blocking and subtraction methods. By way of example only, such substrates may be in the form of dishes, multiwell plates, films, membranes, ribbons, beads, particles, capillary tubes, etc., and maybe chosen to be impermeable or porous to liquids. The techniques used for attachment are chosen for compatibility with the chemical composition of the surface, and may include direct chemical derivatization, covalent crosslinking, indirect coupling such as by antigen-antibody mediated or biotin-avidin bridging, noncovalent coupling by ionic or electrostatic interactions, hydrophobic interactions, copolymerization, gel entrapment, drying, surface tension effects or other techniques known in the art. Suitable procedures for derivatization, for example, may be found by reference to Hermanson in:Bioconjugate Techniques (1996; Academic Press), incorporated herein fully by reference.

If the antigen substrate is a relatively flat surface, then the antigens may be distributed randomly or organized on a grid, so that each position on the grid may be determined or may be placed into registry with the screening monoclonal antibodies or other compounds that are brought into contact. An antigen substrate comprising a cellular “lawn” or monolayer is a convenient format for screening large numbers of candidates. Alternatively, particles and beads may be chosen from among those that impart useful density, magnetic or optical properties to the screening system.

In a preferred embodiment, a PAL is used in conjunction with the screening of monoclonal antibodies generated against immunogens comprising heterogeneous mixtures of potential antigens. It should be understood that heterogeneous mixtures of potential antigens maybe obtained even when a given molecule is defined or cloned, if it is expressed, coupled with or otherwise present among a heterogeneous mixture of other molecules (e.g., intact cells) used for immunization. In preparation for such screening applications, the immunogens used to generate PALs and the immunogen used to generate the monoclonal antibodies being tested can be chosen such that each can generate a range of antigen specificities that may overlap with the specificities of others.

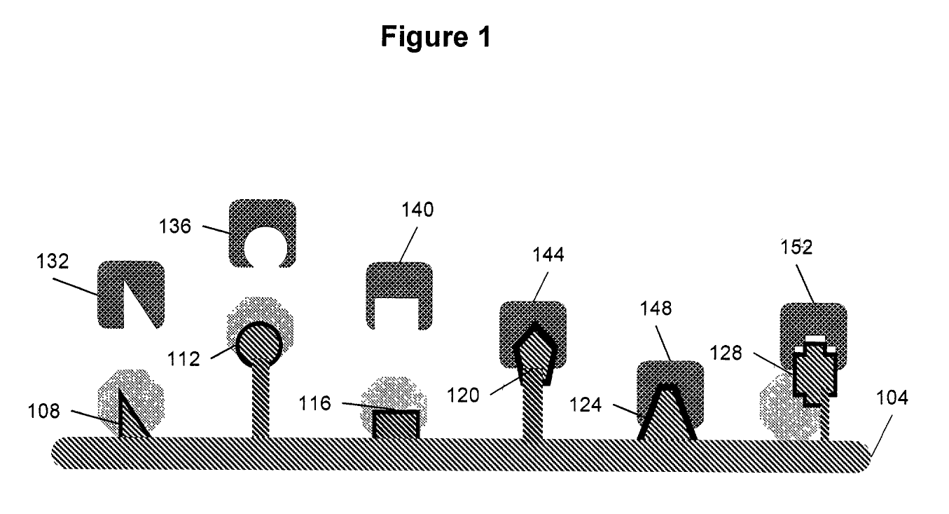

Polyclonal antibody libraries can be used to “mask” antigens in heterogeneous antigen substrates. Components of a PAL can be brought into contact with and can bind to a plurality of different antigens present in the heterogeneous antigen substrate that is then used to screen the test monoclonal antibodies. The PAL thereby can at least partially inhibit or block the subsequent binding of a test monoclonal antibody which has the same or overlapping specificity as those of the PAL. However, the PAL does not substantially interfere with the binding of test monoclonal antibodies directed to the desired antigens not present in the immunogenic antigen mixture used to produce the PAL. These desired antigens may include, for example, antigens with a more restricted expression pattern. An illustration of various outcomes using this approach is depicted in FIG. 1.

FIG. 1 depicts an antigen substrate 100 having a surface 104 with different antigens, 108, 112, 116, 124 and 128 present thereon. Antigens 108, 112 and 116 are masked by antibodies in the PAL antibodies 129, 130, and 131, so that monoclonal antibodies 132, 136 and 140, respectively, are unable to bind to antigens 108, 112 and 116. Antigens 120 and 124 are not masked by antibodies in the PAL, so that test monoclonal antibodies 144 and 148, respectively, are able to bind to antigens 120 and 124. Antigen 128 has several epitopes (sites as determinants at which an antibody may bind), one of which is masked by the antibodies in the PAL, and another of which is not masked and is bound by test monoclonal antibody 152.

By applying suitable detection techniques to differentiate between the test monoclonal antibodies being screened from the bound polyclonal antibodies in the library, the PALS can diminish or, in some cases, abolish signals originating from antigens that are recognized by both the polyclonal antibodies and the test monoclonal antibodies. This antigen subtraction effect has significant benefits for screening monoclonal antibodies.

One way of quantifying the amount of the subtraction effect is through the use of a “signal subtraction ratio” or “SSR.” Signal subtraction ratio is calculated by determining the number of monoclonal antibody clones that react positively with an antigen substrate which is not exposed to the PAL (“unmodified antigen substrate”) divided by the number of monoclonal antibody clones which react positively with an antigen substrate (“modified antigen substrate”) that has antibodies of the PAL bound thereto. An SSR of 1 means that the same number monoclonal antibody clones react positively to the unmodified and modified antigen substrates, and that none of the antibodies in the PAL masks antigens recognized by the test monoclonal antibodies. An SSR of greater than 1 means that at least some of the antibodies in the PAL decrease the binding of test monoclonal antibodies to antigens.

The amount of signal subtraction can be quite substantial. Desired SSR can be in the range of greater than about 1 to over about 1000, alternatively from 1.5 to about 100 and yet alternatively, from about 2 to about 50. In yet further embodiments, the SSR can desirably be in the range of about 5 to about 20, and in still further embodiments, in the range of about 5 to about 15. It can be especially desirable if the SSR is above about 10, although any SSR greater than one represents an improvement in the efficiency of screening monoclonal antibodies. A high degree SSR can be especially desired with complex antigen mixtures such as intact cells. Also, the complex interpretations of immunocytochemical or immunohistochemical staining patterns are simplified by providing quantitative measurement of the degree of subtraction or a simple positive or negative result. In addition, a single positive binding result for a given test monoclonal antibody in the presence of PALs of this invention can replace the results of staining of dozens or more different tissue specimens with that same test monoclonal antibody.

Although not the same a PAL, a narrow version of this blocking principle has been used in an assay method sometimes referred to as “epitope mapping.” In epitope mapping, however, all of the monoclonal antibodies used have predefined specificities for a known antigen molecule, and such assays are generally performed between only two antibodies at one time.

A novel feature of compositions having a PAL is that although the antibodies present have an overall range of reactivity, the specificities of each individual antibody in the PAL need not be known or defined. Antibody libraries are effective in part because the antibody pool can be biased towards common or recurring antigens. The antigens may exist in a wide range of cell types, as well as specificities that may have more restricted ranges of cell distribution.

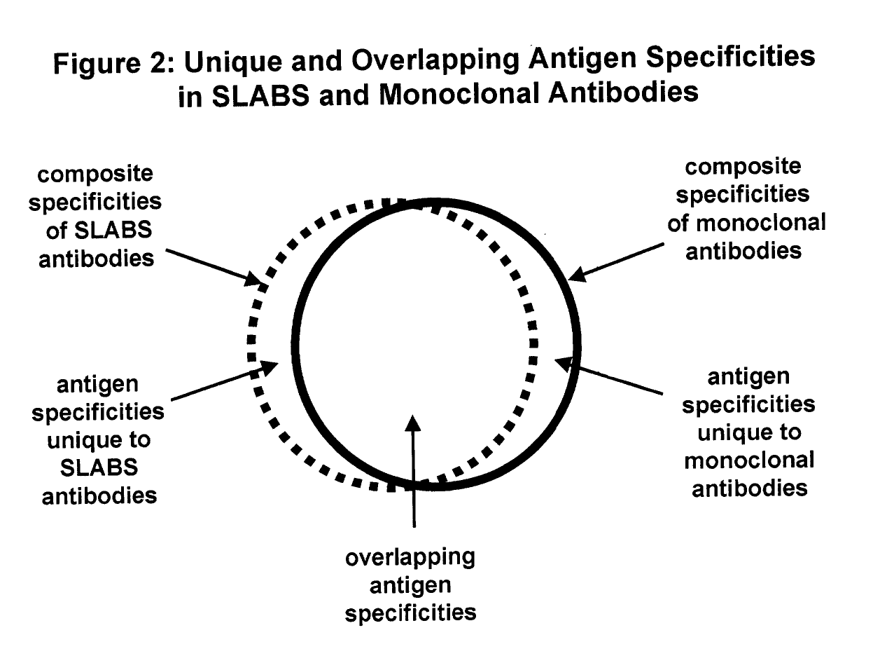

FIG. 2 depicts a schematic example of the range of overlap in antigen recognition that may occur between antibodies in a PAL compared to the totality of monoclonal antibody specificities in a panel of antibodies being screened. Thus, depending on the quality or quantity of the overlapping specificities, a skilled worker in the art may be able to control the level of stringency in the screening to desired effect (e.g., greater overlap may give higher stringency, and vice-versa).

Another novel feature of a PAL is that although it may be a pool or mixture of antibodies that are individually monoclonal in origin, it is not necessary to separately purify or modify the individual monoclonal antibodies comprising the pool, nor is it necessary to clone the individual hybridomas or other cells from which they originate.

An additional novel feature of staging libraries is that they can be a renewable and expandable resource that can replenished by further growing the pooled producer hybridoma cells in vitro or in vivo.

A further novel feature is that the libraries can evolve and be adapted to be more selective by adding monoclonal antibodies or hybridoma cells that are different from antibodies of the original PAL but are not directed against specifically desired antigens derived from further rounds of screening.

Depending on the chosen range of specificities, a PAL may be used for a variety of purposes, for example, to facilitate the discovery and identification of other monoclonal antibodies specific for antigens associated with a desired normal cell lineage, ontogenetic or maturation level or stage, or activation or functional stage. Polyclonal antibody libraries can also be used to help to identify monoclonal antibodies specific for antigens on cells associated with disease processes, for example, different types of tumor cells or stages of malignancy, or cells involved in autoimmune, inflammatory, infectious or other disease states or conditions characterized by antigenic expression. In addition, a PAL can be used to help to identify monoclonal antibodies directed to a distinct epitope on a given antigen. Further, a PAL can be used to help to identify monoclonal antibodies having more desirable binding kinetics or higher affinities to any given antigen.

Some additional nonlimiting examples of the use of PALs for such applications include the following: (a) using a PAL made to mature cells in different lineages when screening for monoclonal antibodies to antigens on stem cells or precursor cells for those lineages; (b) screening of monoclonal antibodies to antigens on activated cells or cells involved in autoimmune or inflammatory disease processes e.g., T or B lymphocytes, killer cells, dendritic cells or other antigen-presenting cells (“APC”), regulatory lymphocytes) using a PAL made to the non-activated or normal cell counterparts; (c) using a PAL made to antigens of a less virulent strain of an infectious microorganism, e.g., a bacterium, virus or protozoan, when screening for monoclonal antibodies to antigens on other strains associated with greater virulence; and (d) making a PAL to a known antigen in order to screen for monoclonal antibodies to a distinct epitope or that bind with higher affinity (i.e., by displacing lower affinity antibodies with similar specificity).

The production of PALs may employ any method known in the art for raising antibodies to complex mixtures of antigens, including intact cells, cell extracts or fractions. Polyclonal antibody libraries can also be made using phage display antibody methods. The nature of the immunogen may be varied to obtain the desired level of specificity or stringency for the baseline antigen subtraction with the staging libraries. For example, to screen for monoclonal antibodies specific for novel antigens on cancer cells, it may be desirable to make PALs with specificities for the normal cell lineage from which the cancer arose. The PALs so made can then used to modify an antigen substrate containing the potential cancer antigens before screening the monoclonal antibodies on the substrate. Using prostate cancer as an example that is applicable to any other type of tumor, a PAL made against normal prostate tissue can be used when screening for monoclonal antibodies to prostate tumor specific antigens. A more stringent example in the cancer field might be to make PALs for staging using primary tumor cells as immunogens and to screen monoclonal antibodies against metastatic cells of the same tumor (or vice-versa).

For any of these purposes, the cells used for the immunization or for producing the antigen substrate, whether as intact cells, cell extracts or fractions, organelles, digests, and the like, can be obtained from commonly available resources and culture techniques, or they can be modified by recombinant techniques.

In alternative embodiments, to increase an immune response of an antibody producing cell, cells of the immunogen can be transformed and cultures expanded to provide a large pool of immunogen. A variety of both normal and malignant cell types are available from commercial or government suppliers and/or repositories (e.g., American Type Culture Collection or “ATCC”; National Institutes of Health Research and Reference Reagent Programs) as primary tissue or established or transformed lines.

Genetic modification of cells for such purposes may involve, for example, upregulating or downregulating a particular gene, or transfecting a host cell line with a single gene or CDNA, a collection of genes or an entire cDNA library such as a subtraction library. For example, suitable host cells for transfection include lines available from the ATCC such as: human cervical carcinoma cells (HELA); human lung cells (W138); human liver cells (Hep G2); human embryonic kidney line (293); monkey kidney cells (CV1); monkey kidney CVI line transformed by SV40 (COS-7); baby hamster kidney cells (BHK); chinese hamster ovary-cells-DHFR (CHO); african green monkey kidney cells (VERO-76); canine kidney cells (MDCK); buffalo rat liver cells (BRL 3A; mouse mammary tumor (MMT). For these applications the term “host cell” refers to those vertebrate cells capable of growing in culture and expressing desired antigen(s). While the preferred host cells of this invention are vertebrate cells, other eukaryotic cells may be used, such as for example, insect cells. To detect antigens that arise from infection by procaryotic cells (e.g., bacteria) or viruses, PALs can be made to both uninfected target cells and the prokaryotic cell or virus. Thus, a staging library can be prepared that can react with both the normal cellular antigens and pathogen's antigens. Then, testing target cells infected with the bacterial (or virus) can permit detection of cell and/or pathogen-specific antigens produced as a result of the infection.

Techniques for transfecting or transducing genetic material into such lines are described in numerous laboratory methods manuals known in the field of molecular biology. These include, for example: electroporation, calcium phosphate precipitation, liposomal vectors, synthetic vectors, adenoviral vectors, and retroviral vectors.

Alternatively, intact cells may be used as carriers or indicators for exogenously obtained antigens by coupling them with haptens, peptides, soluble proteins or extracts or fractions from other cells. The carrier cells used for this purpose may be nucleated or non-nucleated (e.g., mammalian erythrocytes). A variety of methods are known in the art for accomplishing such couplings, including, for example, direct chemical conjugation, using chemical crosslinkers, or biotin-avidin bridging.

The process for producing a PAL may employ the same initial methods known in the art for making monoclonal antibodies. Using the preferred approach of generating hybridomas, the lymphoid organs of the immunized animals are harvested and fused with parental myeloma cells as referenced above. The hybridomas are then subjected to drug selection (e.g., commercially available, standard HAT-containing medium) to eliminate unfused myeloma cells. However, unlike traditional hybridoma methods where the cells are drug selected following seeding in limiting dilution culture in microwells, the cells for producing staging libraries can be drug selected collectively while they are in batch culture. The cells may then be expanded in culture or in ascites for the purpose of obtaining useful quantities of secreted polyclonal antibodies.

It may be desirable to prevent a reduction in overall antibody specific activity (e.g., concentration or quantity), or to increase the activity of relevant antibodies in the culture medium or ascites fluid. Activity may be diminished by overgrowth of nonsecreting hybridoma cells or cells secreting irrelevant or incomplete immunoglobulin molecules, for example. It is commonly known that overgrowth of undesired populations can be reduced by early expansion of cell lines and cryopreserving in aliquots for future limited-duration use, rather than by continuous serial passaging. In addition, methods are known in the art of cell immunoselection that could be adapted to recover hybridoma cells that continue to produce immunoglobulin molecules from those presumptive non-antibody producing cells that do not.

Selecting hybridoma cells as a source of a PAL can be based on the generic production of immunoglobulin molecules in the form of residual cell surface immunoglobulin (i.e., antibody receptor for antigen), and can be accomplished by techniques such as staining the antibody with a complementary fluorescent anti-immunoglobulin antibody and then sorting by flow cytometry. Alternatively, one can select cells by labeling the antibody with complementary anti-immunoglobulin antibody linked to immunomagnetic beads (e.g., Dynal Biotech, Oslo, Norway; Miltenyi Biotec, Auburn, Calif.) and then magnetic cell sorting. It may be noteworthy that such cell surface immunoglobulin is considered to represent residual membrane bound antigen receptor antibody from an earlier lymphoid or lymphoblast maturation stage that is not bears no correlation with the amount of antibody that is secreted from a hybridoma or plasma cell. It can be readily appreciated that any other methods for selecting antibody producing cells can be used to provide staging libraries.

In a preferred embodiment, the specific activity of a PAL may be increased further, and in certain embodiments, can approach 100 percent antigen-specific antibodies. Increasing specific activity can be accomplished by immobilizing the secreted antibodies monoclonally onto the surface membranes of the respective hybridoma or other cells of origin, and then affinity-selecting these cells by adhesion to an antigen substrate. The antigen of the substrate for this method can be typically of the same or similar antigen composition at the immunogen used to generate the PAL. This type of selection by specificity can be carried out around the same time (i.e., before, during, or after) as the batch HAT (or other drug) selection or a later time of cell culture or expansion.

Methods and compositions for this and other purposes are described in an accompanying patent application titled “Methods for Screening Antibody-Producing Cells on Heterogeneous Antigen Substrates” Steven Kessler, inventor, Attorney Docket No. KSLR 1001 USO SRM/DBB, filed concurrently, herein incorporated fully by reference.

Once suitable quantities of antibodies are produced into culture medium or ascites, the antibodies can be collected and manipulated in accordance with a chosen method that serves to distinguish these antibodies from the monoclonal antibodies to be screened. Such distinctions may be based on antigenic or structural markers on the immunoglobulins of different animal species, classes or isotypes, subclasses, allotypes, or sizes using commercially available antibodies or other reagents for this purpose. For example, human monoclonal antibodies being screened may be distinguished from a PAL consisting of murine antibodies by use of secondary or indirect immunofluorescence or immunoenzymatic staining involving anti-human immunoglobulin reagents. Other usable labels besides fluorescers and enzymes include radiosiotopes, chemiluminescers, phosphors, particles, etc. In addition, measurements of affinities, avidities, association rates or dissociation rates can be made directly or indirectly on the monoclonal antibodies by surface plasmon resonance (e.g., BIAcore).

A preferred embodiment when PALs and screening monoclonal antibodies originate from the same animal species involves the conversion of PALs to antibody fragments and the concurrent use of secondary or indirect immunofluorescence or immunoenzymatic staining reagents that bind to the screening monoclonal antibodies in their structurally intact form. Antibody fragments comprise a portion of a full length antibody, generally the antigen binding or variable region thereof, and lack all or part of the Fc region. Examples of antibody fragments include Fab, Fab′, F(ab′)₂, Fv fragments and single-chain antibody molecules. Fc-specific anti-immunoglobulin class antibodies (such as sheep anti-mouse IgG-Fc, as a non-exclusionary example) or protein A are examples of commercially available reagents that could serve this purpose. Reagents and kits are also available commercially for preparing antibody fragments and purifying them away from intact antibodies (e.g., from Pierce Chemical Co., Rockford, Ill.).

Ideally, the reaction between the constituent antibodies in a PAL and the antigen substrate is carried out under conditions in which the antibodies saturate or block all of the antigenic sites on the substrate for which they have specificity. Experimental measures used separately or in combination to help accomplish this include raising the total protein concentration of the staging libraries, and thus the concentrations of the individual constituent antibodies. Empirical calculations indicate that protein concentrations needed to achieve this (typically 1-5% w/v or less,) can be obtained in the laboratory.

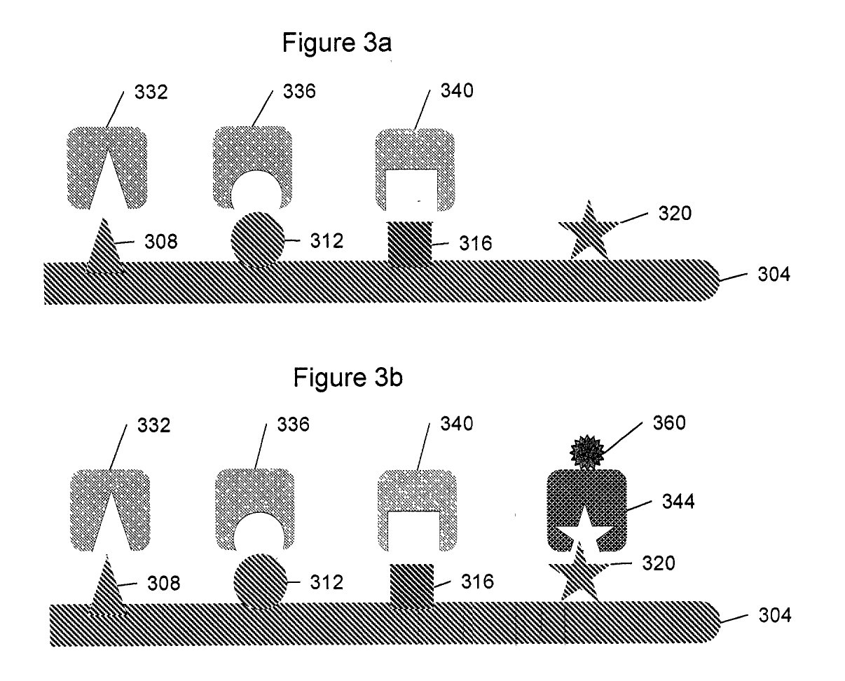

FIGS. 3a and 3b depict schematically an embodiment of this invention 300, having surface 304 and a heterogeneous antigen substrate having antigens 308, 312 and 316 present on a surface 304. Antibodies 332, 336 and 340 in a PAL interact with and mask their respective antigens 308, 312 and 316. In FIG. 3a, the additional antigen 320 in the substrate is not masked. In FIG. 3b, monoclonal antibody 334 is additionally bound to antigen 320, and is detected by labeling with a reporter. Reporters suitable for use include fluorophores, enzymes, radionuclide or bead coupled to another antibody or labeling reagent.

Other useful measures favoring saturation binding include diluting the antigen concentration on the substrate, lowering the temperature during the interaction to reduce antibody dissociation rate, conducting the monoclonal antibody screening assay in the presence of excess PAL rather than washing it out beforehand, and/or covalently attaching staging library antibodies to the antigen to prevent dissociation. Covalent attachment offers an advantage in allowing PAL-treated antigen substrates to be prepared hours, days or weeks in advance of monoclonal antibody screening. A variety of homobifunctional and heterobifunctional chemical crosslinking reagents and kits are available commercially (e.g., from Pierce Chemical Company) that can be used for this purpose including, but not limited to those containing aldehydes, succinimidyl esters, imidoesters, maleimidyl esters, hydrazides, aryl azides, and carbodiimides.

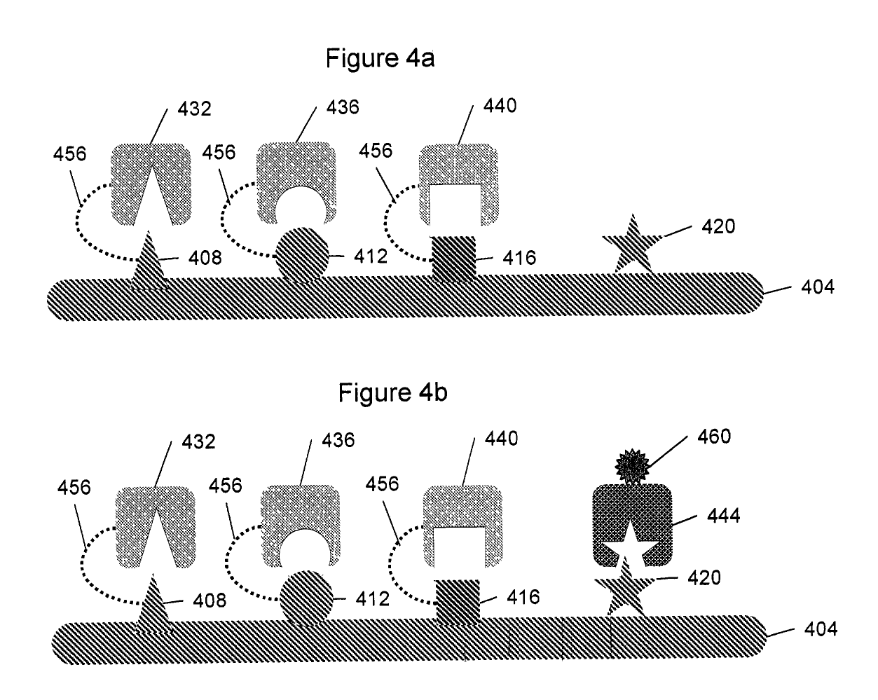

FIGS. 4a and 4b depict schematically an embodiment of this invention 400 having a substrate 404, with antigens 408, 412, 416 and 420 thereon. Antibodies 432, 436 and 440 in a PAL interact with and are then covalently attached to their respective antigens present in a heterogeneous antigen substrate with cross-linkers 456. In FIG. 4a, antigen 420 is not masked by antibodies in the PAL. In FIG. 4b, the monoclonal antibody 444 is bound to antigen 420, and has reporter 460 attached to antibody 444. Reporter 460 permits detection of antibody 444. Reporters can include fluorophores, enzymes, radionuclides and/or beads coupled to another antibody or labeling reagent.

The ability to provide stable PAL-treated antigen substrates can permit the construction of kits suitable for a variety of uses. By way of example only, kits can be provided that comprise PAL-producing cells in frozen form. Expansion of PAL-producing cells can provide a renewal source of staging antibodies for a method for detecting novel antibodies by antigen subtraction using “staging library for antigen binding and subtraction” or “SLABS™” . Additionally, a PAL can be in the form of partially or more completely purified antibodies. Such preparations can be obtained from hybridoma cell supemates or ascites. A PAL may be stored in liquid medium, or may be lyophilized to form a more stable powder. Other kit embodiments include substrates comprising a heterogeneous antigen mixture associated therewith “heterogeneous antigen substrates,” and a separate preparation including one or more PALs. Alternatively, a kit may contain one or more heterogeneous antigen substrates with a PAL bound thereto, either covalently or non-covalently. In yet other embodiments, a kit may comprise heterogeneous antigen mixtures derived from a variety of different cell types, including normal cells, diseased cells, or pathogens that can infect or transform normal cells, fragments thereof, and the like.

One or more of the aforementioned methods may find use in the context of screening monoclonal antibodies originating from hybridoma cells. However, it can be readily appreciated by those skilled in the art that methods of this invention may be applied more broadly to any antibody that is diffusible or secreted in monoclonal form and capable of binding to an antigen substrate. The origins of such antibodies may include bacteria, bacteriophage, yeast, other eucaryotic cells genetically modified to produce antibodies (e.g., transfectomas), or single non-immortalized primary antibody-forming or plaque-forming cells or plasma cells.

A technique for detecting a specifically bound antibody that is physically associated with the producing cell of origin, such as bacteriophage, may include labeling with an antibody for some other known antigen on the cell rather than the bound antibody itself. Basic methods for constructing and screening antibody phage display libraries are described by McCafferty and Johnson (1996) in: Phage Display of Peptides and Proteins (Kay, Winter, McCafferty, eds., Academic Press, pp. 79-111, which is herein incorporated fully by reference.

Primary antibody-forming or plaque-forming cells may be detected by the formation of zones of lysis of erythrocytes coated with antigens in the presence of complement, using a standard hemolytic plaque assay. In such an example, an antigen substrate may be applied to a surface as a cell suspension, typically as a monolayer, that is fixed in place by surface tension or by inclusion in a gelling medium. Variations of the plaque assay method are known or can be devised by those skilled in the art that utilize intact nucleated cell targets in place of erythrocytes. An antibody producing cell may then be isolated from the centers of these zones by micromanipulation. Such methods can be applied to the extraction of either mRNAs or genomic DNAs encoding the specific antibody genes from antibody forming cells or hybridoma cells, using techniques similar to those described by Babcook et al. (1996) Proc. Natl. Acad. Sci. USA 93:7843), herein incorporated fully by reference.

The following synopsis of a monoclonal antibody screening process using a PAL is offered as an example and not by way of limitation.

### EXAMPLES

### Example 1

An epitope mapping experiment was performed to demonstrate a narrow version of the principle of using antibodies to block certain epitopes on a cell surface antigen molecule, while allowing the binding of antibodies to other epitopes. For this purpose, four monoclonal antibodies of mouse origin were used (designated as Fabre, GM201, SM41b, and 5E10; generously provided by SyStemix, Inc., Palo Alto, Calif./Novartis Pharmaceutical Corp., Basel, Switzerland and used with permission) that were known to bind to the human Thy-1/CD90 molecule that is expressed on hematopoietic stem cells and certain immature T cells (Leucocyte Typing Workshop VII, Mason D et al., eds. (2000) Oxford University Press; The Leucocyte Antigen FactsBook, Barclay A N et al., eds. (1993) Academic Press). It was of general technical interest to determine if the relatively small size of the Thy-1 cell surface molecule (mol. wt. ca. 18,000), compared to antibody molecules (mol. wt. ca. 150,000), would limit the ability to detect more than one epitope.

The anti-Thy-1 monoclonal antibodies produced by the respective hybridomas were purified from cell culture or ascites fluids by protein A chromatography, and portions of the antibody preparations were conjugated with either fluorescein (using fluorescein-NHS, Calbiochem, San Diego, Calif.) or biotin (using biotin-NHS; Calbiochem).

Separate aliquots of the human Jurkat T cell line (obtained from the ATCC) were preincubated with high saturating amounts (200-500 ug/ml; concentrations determined empirically) of each of the unconjugated anti-Thy-1 monoclonal antibodies listed in Table 1 below as “Blocking Antibody” (or an irrelevant IgG preparation as a positive control), and then incubated with a nominal amount of fluorescein or biotin conjugate of one of the other MAbs listed in the table as “Test Antibody” (or the same antibody as a negative specificity control). If a biotin conjugate was used, then the cells were secondarily stained with a fluorescein-streptavidin conjugate. The amount of inhibition of cell fluorescence staining intensity was then analyzed by flow cytometry (FACS, Becton-Dickinson, San Jose, Calif.), as indicated on the table. If no inhibition was seen, then it was clear that the blocking and test antibodies were each binding to distinct epitopes on the Thy-1 molecule.

The results in Table 1 suggest at least two distinct epitope clusters represented by GM201 vs. SM41b and 5E10. SM41b, 5E10 and Fabre each appear to represent adjacent but separate epitopes. By analogy to the principle of PALs, if antibodies Fabre, SM41b and 5E10 had been components of a PAL of unknown specificity masking epitopes of an unknown antigen represented by Thy-1, then test antibody GM201 would have been revealed as binding to a novel epitope.

TABLE 1
Thy-1 Epitope Mapping

| Blocking | Level of Inhibition of Test Antibody* | | | |
|---|---|---|---|---|
| Antibody | High | Medium | Low | None |
| Fabre | Fabre, SM41b | 5E10 | | GM301 |
| GM201 | GM201 | | Fabre | SM41b, 5E10 |
| SM41b | SM41b, Fabre | 5E10 | | GM201 |
| 5E10 | 5E10, Fabre | SM41b | | GM201 |

*Inhibition Levels:
High - background level or 10-20-fold reduction in staining intensity;
Medium - 2-5-fold reduction in staining intensity;
Low - 1-2-fold reduction in staining intensity;
None - no effect on staining intensity.

### Example 2

With the intention of screening for monoclonal antibodies to antigens specific for prostate tumor cells, groups of mice are immunized with either normal prostate-derived cells or prostate tumor cells. Hybridomas are generated from the lymphoid organs of the normal cell-immunized mice, HAT selected and expanded collectively in batch culture, and 10-20 liters of conditioned medium are produced. The IgG antibodies are purified by protein A chromatography, digested to Fab′ fragments, F(ab′)₂, further purified according to methods known in the art and concentrated to about 5-50 mg/ml of protein.

Hybridomas are similarly generated from the lymphoid organs of the tumor cell-immunized mice, seeded in limiting dilution into microwell culture plates, and HAT selected. Approximately 9-12 days later the monoclonal antibodies contained in the culture medium are collected from the individual wells for screening.

Prostate tumor cells are seeded into petri dishes or multiwell plates to establish the antigen substrate. The cells are grown until they reach a state of near-confluency. Alternatively, they are grown elsewhere and then attached using the adhesion promoting chemical polylysine. After removal of nonadherent cells by washing, an aliquot of the SLABS antibody solution is added to each well and incubated for a minimum of one hour. The substrate is then ready for screening with the anti-prostate tumor monoclonal antibodies.

Alternatively, the staging antibodies are covalently crosslinked to their respective antigens on the substrate so that it can be used the following week. The chemical crosslinking agents glutaraldehyde, disuccinimidyl suberate or a water-soluble carbodiiumide are used to equal effect. The crosslinker is then washed away and the substrate stored until needed.

An aliquot of culture medium containing each monoclonal antibody that is to be screened is then added to the individual treated antigen microwells, which are numbered to correspond to the hybridoma wells of origin. After additional incubation, the cells are washed by changes of medium, and an aliquot of horseradish peroxidase conjugated sheep anti-mouse IgG-Fc antibody is added. After additional incubation, the cells are washed further and peroxidase substrate is added and incubated. Monoclonal antibodies from wells showing evidence of positive color reaction are considered to be specific for a unique or tumor associated antigen.

### INDUSTRIAL APPLICABILITY

Methods, devices and compositions of this invention can be used to screen monoclonal antibodies for medical, diagnostic, and experimental purposes. Polyclonal antibody libraries can be used to mask undesired antigens in mixed antigen substrates, thereby increasing the efficiency of screening of antibodies directed toward desired antigens. The methods, compositions and devices can decrease the cost and time required to screen and/or evaluate antibodies for a variety of uses.

1. A method for manufacturing a library of polyclonal antibody library-producing cells, comprising the steps of:
(a) immunizing an animal with an immunogen comprising a mixture of antigens not having an antigen of interest;
(b) obtaining a mixture of antibody producing cells from said animal, said antibody producing cells producing antibodies directed toward at least a portion of said antigens from said immunogen; and
(c) immortalizing said antibody producing cells.

2. The method of claim 1, wherein said immunogen comprises antigens selected from the group consisting of cells, transfected cells, cell fragments, cellular ogranelles, cell fractions, cellular digests, cellular molecules and molecular digests.

3. The method of claim 1, where said step of immortalizing is carried out in vitro.

4. The method of claim 3, wherein said step of immortalizing includes producing hybridomas.

5. The method of claim 4, wherein said hybridomas are subjected to drug selection.

6. The method of claim 5, wherein said drug selection is carried out using hypoxantine, aminopterin, thymnidine (HAT)-containing medium.

7. The method of claim 1, further comprising the step of collecting said antibodies, forming a polyclonal antibody library (PAL).

8. The method of claim 7, wherein said antibody library contains at least one functional antibody moiety selected from the group consisting of antibody fragments, Fab, Fab′, F(ab′)₂, Fv fragments, single-chain and/or single domain antibody molecules, and recombinant antibodies.

9. The method of claim 8, wherein said function is determined by binding of said antibody to an antigen.

10. The method of claim 1, wherein said polyclonal library-producing cells are produced by transfection of antibody genes into another cell.

11. The method of claim 1, wherein said polyclonal library-producing cells are produced by expression of antibody moieties using filamentous phage vectors.

12. The method of claim 1, wherein at least one of said antigens is from a eucaryotic cell.

13. The method of claim 1, wherein at least one of said antigens is from a procaryotic cell.

14. The method of claim 1, wherein at least one of said antigens is from a virus.

15. The method of claim 12, wherein said eucaryotic cell is derived from at least one origin selected from the group consisting of ectodermal, endodermal and mesodermal origin.

16. The method of claim 1, wherein at least one of said antigens is selected from the group consisting of primary cells, cell lines and immortalized cells that retain a normal cell antigen phenotype.

17. The method of claim 1, wherein at least one of said antigens is from a normal cell selected from the group consisting of breast, ovary, prostate, colorectal, lung, brain, kidney, pancreas, skin, connective tissue, intestinal, muscle, or hematologic cells.

18. The method of claim 1, wherein at least one of said antigens is from a tumor cell.

19. The method of claim 18, wherein said cell is selected from the group consisting of a metastatic tumor cell and a primary tumor cell.

20. The method of claim 1, wherein at least one of said antigens is from a cell is selected from the group consisting of normal mature cells and cells from tissues of a known lineage.

21. The method of claim 20, wherein said cell is a lymphocyte selected from the group consisting of immature lymphocytes, mature lymphocytes and differentiated lymphocytes.

22. The method of claim 20, wherein said lymphocyte is not activated.

23. The method of claim 20, wherein said lymphocyte is selected from the group consisting of T-lymphocytes and B-lymphocytes, killer cells, dendritic cells and dendritic cells.

24. The method of claim 1, wherein at least two of said antibodies react to different epitopes of the same antigen molecule.

25. A polyclonal antibody library (PAL), comprising a plurality of monoclonal antibodies derived from a renewable source, at least one of said antibodies not directed toward an antigen of pre-defined specificity.

26. The PAL of claim 25, wherein said renewable source is an immortalized cell culture.

27. The PAL of claim 25, which comprises the collected, purified or concentrated secreted antibodies.

28. The PAL of claim 25, wherein said antibodies are selected from the group consisting of igG, IgE, IgA, IgD and IgM antibodies.

29. The PAL of claim 25, wherein said PAL comprises antibody fragments, including Fab, Fab′, F(ab′)₂, Fv fragments, single-chain antibody molecules and single-domain antibody molecules, and recombinant antibodies.

30. The PAL of claim 25, wherein said renewable source comprises immortalized hybridoma cells.

31. The PAL of claim 30, wherein said hybridoma cells are subjected to drug selection and maintained as batch cultures.

32. The PAL of claim 25, wherein said renewable source comprises cell lines transfected with antibody genes.

33. The PAL of claim 32, wherein said renewable source comprises cell lines transfected with phage antibody libraries.

34. A method for identifying monoclonal antibodies, comprising the steps of:
(a) providing a heterogeneous antigen substrate (“HAS”);
(b) exposing said HAS to a PAL directed towards said HAS, said PAL associating with at least one antigen of said HAS, producing a PAL-treated HAS;
(c) applying a test monoclonal antibody to said PAL-treated HAS; and
(d) detecting binding of said monoclonal antibody to an antigen on said HAS.

35. The method of claim 34, wherein the antigen substrate consists of a cellular lawn.

36. The method of claim 34, wherein said HAS comprises a plurality of addressable locations.

37. The method of claim 35, wherein the cellular antigen substrate is eucaryotic.

38. The method of claim 34, wherein said HAS is derived from procaryotic cells.

39. The method of claim 34, wherein said monoclonal antibodies are made against a member of the group consisting of tumors of breast, ovary, prostate, colon, rectum, lung, brain, kidney, and a blood cell, and wherein said PAL is made against a normal cell or tissue counterpart from which monoclonal antibodies directed at said tumor arose.

40. The method of claim 34, wherein said monoclonal antibodies are directed towards metastastized tumor tissue and said PAL is made against primary tumor tissue.

41. The method of claim 34, wherein said PAL is directed against a metastastized tumor tissue and said monoclonal antibody is directed towards a primary tumor tissue.

42. The method of claim 34, wherein said PAL are made against normal cells and said monoclonal antibodies are made against precursor cells of the lineage producing said normal cells.

43. The method of claim 34, wherein said monoclonal antibodies are made to antigens on an activated cell and the PAL is made to a non-activated counterpart of said activated cell.

44. The method of claim 43, wherein said activated cell is a cell involved in a disease selected from autoimmune and inflammatory diseases.

45. The method of claim 44, wherein said cell is selected from the group consisting of T lymphocytes and or B lymphocytes.

46. The method of claim 34, wherein said monoclonal antibodies and said PAL react against different epitopes of the same antigen molecule.

47. The method of claim 34, wherein said monoclonal antibodies and said PAL bound to the heterogeneous antigen substrate have one of antigenic and structural differences.

48. The method of claim 47, wherein said difference is selected from the group consisting of corresponding to immunoglobulin animal species, classes, isotypes, subclasses, allotypes, and sizes.

49. The method of claim 34, wherein said PAL comprises Fab, Fab′, F(ab′)₂, Fv fragments and single-chain antibody molecules, and the monoclonal antibodies comprise structurally intact molecules having Fc portions.

50. The method of claim 34, wherein the step of detecting includes using Fc-specific anti-immunoglobulin antibody, protein A, or protein G.

51. The method of claim 34, wherein said step of detecting includes using fluorescers, enzymes, radiosiotopes, chemiluminescers, phosphors, particles, beads, membranes and plasmon resonance.

52. The method of claim 34, wherein said step of detecting includes using an assay for hemolytic plaques.

53. The method of claim 34, wherein said step of detecting includes detecting a zone of labeling of target cells surrounding antibody-forming or plaque-forming cells.

54. The method of claim 34, wherein said monoclonal antibodies are expressed on the surfaces of bacteriophage.

55. The method of claim 42, which comprises an antigen substrate bound with antibodies from a PAL.

56. The method of claim 47, which comprises an antigen substrate bound with covalently attached antibodies from a PAL.

57. The method of claim 49, which comprises an antigen substrate bound with antibody fragments from a PAL.

58. The method of claim 49, which comprises an antigen substrate bound with covalently attached antibody fragments from a PAL.

59. A method of manufacturing a PAL comprising the steps of:
(a) immunizing an animal with an immunogen comprising a mixture of antigens not having an antigen of interest;
(b) obtaining a mixture of antibody producing cells from said animal, said antibody producing cells producing antibodies directed toward at least a portion of said antigens from said immunogen;
(c) immortalizing said antibody producing cells; and
(d) collecting said antibodies.

60. A kit, comprising:
a heterogeneous antigen substrate; and
a PAL directed towards said heterogeneous antigen substrate.

61. The kit of claim 60, wherein said PAL is provided in a form selected from the group consisting of lyophilized, soluble, a solution and frozen.

62. The kit of claim 60, wherein said PAL is associated with said heterogeneous antigen substrate.

63. The kit of claim 62, wherein said association is by way of covalent cross-links.

64. The kit of claim 60, further comprising:
at least one monoclonal antibody; and
a reference reagent.

65. The kit of claim 64, further comprising a reporter.

66. The method of claim 1, wherein at least one of said antigens is from a cell infected with a virus.

67. The method of claim 1, wherein at least one of said antigens is from a cell infected with a microorganism selected from the group consisting of bacteria, protozoa, and fungi.

68. The method of claim 1, wherein said polyclonal library-producing cells are produced from yeast.

69. The PAL of claim 25, wherein said renewable source is a filamentous bacteriophage library.

70. The PAL of claim 25, wherein said renewable source is a yeast cell library.
